(19) United States
(12) Patent Application Publication
(10) Pub. No.: US 2020/0368122 A1
(43) Pub. Date: Nov. 26, 2020

(54) COMPOSITION FOR PREVENTING HAIR LOSS AND PROMOTING HAIR GROWTH AND METHOD FOR PREPARING THE SAME

(71) Applicant: MEDIPOOL CO., LTD., Gyeongsan-si, Gyeongsangbuk-do (KR)

(72) Inventor: Ho Cheong KIM, Daegu (KR)

(21) Appl. No.: 16/965,214

(22) PCT Filed: Mar. 6, 2019
(86) PCT No.: PCT/KR2019/002596

§ 371 Date: Jul. 27, 2020

(30) Foreign Application Priority Data
Mar. 12, 2018 (KR) 10-2018-0028432

(51) Int. Cl. A61K 8/19; A61K 8/34; A61K 8/97; A61K 8/02; A61Q 7/00

(52) U.S. Cl. CPC A61K 8/19; A61K 8/345; A61K 8/97; A61K 2800/80; A61Q 7/00; A61K 2800/413; A61K 2800/59; A61K 8/0241

(57) ABSTRACT

A composition for preventing hair loss and promoting hair growth includes: 100 parts by weight of an aqueous colloid solution containing nonionic copper nanoparticles having an average particle diameter (D50) of about 2 nm to about 10 nm; about 1 part by weight to about 10 parts by weight of glycerin; and about 1 part by weight to about 10 parts by weight of a co-solvent, wherein the nonionic copper nanoparticles are present in a concentration of about 2,000 ppm to about 3,000 ppm. The composition has the advantages of not only preventing hair loss but also promoting hair growth.

### TECHNICAL FIELD

The present invention relates to a composition for preventing hair loss and promoting hair growth and a method for preparing the same.

### Background Art

Hair loss refers to increase in hair that is abnormally missing as the proportion of hair growing in hair follicles in a degenerative or resting period increases rather than hair growing in the hair follicles during a growth period of the hair follicles. In recent years, persons complaining of hair loss are increasing due to various types of stress and environmental pollution. As a result, production and consumption of air loss-related products are also increasing.

Although various factors, such as poor blood circulation, male hormone hyperactivity, sebaceous hypersecretion, scalp hypoplasia due to peroxides and bacteria, genetic factors, aging, and stress, have been discussed as the cause of hair loss, the exact cause of hair loss is still unknown. In general, the most typical form of hair loss is male alopecia caused by male hormone hyperactivity and is dependent on androgen which is a male hormone. Typical androgens include testosterone and dihydrotestosterone (DHT), and alopecia patients are known to have more dihydrotestosterone. Reports say that testosterone is converted into dihydrotestosterone by 5α-reductase, specifically by Type 2 5α-reductase among 2 types of 5 α-reductase including Type 1 and Type 2 5α-reductases, and gradual reduction of dermal papilla and hair follicles is carried out by the converted dihydrotestosterone.

In treatment and prevention of such alopecia, current methods cause side effects on the skin or provide insignificant effects in prevention of hair loss and boosting hair growth.

### DISCLOSURE

### Technical Problem

It is one object of the present invention to provide a composition for preventing hair loss and promoting hair growth, which can prevent hair loss while securing good effects in boosting hair growth, and a method for preparing the same.

It is another object of the present invention to provide a composition for preventing hair loss and boosting hair growth, which does not have side effects even after use for a long period of time, and a method for preparing the same.

The above and other objects of the present invention can be achieved by the present invention described below.

### Technical Solution

One aspect of the present invention relates to a composition for preventing hair loss and promoting hair growth.

In one embodiment, the composition for preventing hair loss and promoting hair growth includes: 100 parts by weight of an aqueous colloid solution containing non-ionic copper nanoparticles having an average particle diameter (D50) of about 2 nm to about 10 nm; about 1 part by weight to about 10 parts by weight of glycerin; and about 1 part by weight to about 10 parts by weight of a co-solvent, wherein the non-ionic copper nanoparticles are present in a concentration of about 1,500 ppm to about 2,500 ppm in the aqueous colloid solution.

In another embodiment, the aqueous colloid solution may further contain about 20 ppm to about 200 ppm of silver nanoparticles.

The copper nanoparticles and the silver nanoparticles may be present in a concentration ratio of about 10:1 to about 50:1.

In a further embodiment, the composition may further include about 0.01 parts by weight to about 3 parts by weight of a combined extract of Coicis semen and Rehmannia glutinosa.

Another aspect of the present invention relates to a method for preparing a composition for preventing hair loss and promoting hair growth.

In one embodiment, the method for preparing a composition for preventing hair loss and promoting hair growth includes: preparing an aqueous colloid solution containing non-ionic copper nanoparticles having an average particle diameter (D50) of about 2 nm to about 10 nm; and adding glycerin and a co-solvent to the prepared aqueous colloid solution, followed by uniformly mixing the glycerin and the co-solvent with the aqueous colloid solution, wherein the aqueous colloid solution is prepared by adding sodium hydroxide (NaOH) to an aqueous copper chloride (CuCl₂) solution to generate copper oxide and copper hydroxide in the aqueous solution and adding hydrazine (N₂H₄) to the aqueous solution to reduce the copper oxide and the copper hydroxide into non-ionic copper nanoparticles.

The aqueous colloid solution may include about 1,500 ppm to about 2,500 ppm of the non-ionic copper nanoparticles, the sodium hydroxide (NaOH) may be added in an amount of about 1 mole to about 6 moles per mole of copper chloride (CuCl₂), and the hydrazine (N₂H₄) may be added in an amount of about 1 mole to about 12 moles per mole of copper chloride (CuCl₂).

The aqueous colloid solution may further include about 20 ppm to about 200 ppm of silver nanoparticles and the copper nanoparticles and the silver nanoparticles may be present in a concentration ratio of about 10:1 to about 50:1.

The method may further include: uniformly mixing a combined extract of Coicis semen and Rehmannia glutinosa with the aqueous colloid solution, the combined extract being extracted using a solvent containing about 90 wt % to about 99 wt of ethanol and about 1 wt % to about 10 wt % of glycerin.

### Advantageous Effects

The present invention provides a composition for preventing hair loss and promoting hair growth, which can prevent hair loss while securing good effects in boosting hair growth and does not have side effects even after use for a long period of time, and a method for preparing the same.

### BRIEF DESCRIPTION OF DRAWINGS

FIG. 1 is a scanning electron microscope (SEM) image of non-ionic copper nanoparticles contained in a composition for preventing hair loss and promoting hair growth prepared in Example 1.

### BEST MODE

Hereinafter, embodiments of the present invention will be described in detail. It should be understood that the following embodiments are provided for illustration and are not construed in any way as limiting the present invention.

In description of the present invention, descriptions of known functions and constructions which can unnecessarily obscure the subject matter of the invention will be omitted.

Herein, the terms “comprises” and/or “comprising,” when used in this specification, specify the presence of stated features, steps, operations, elements, and/or components, but do not preclude the presence or addition of one or more other features, steps, operations, elements, components, and/or groups, unless otherwise defined herein,. In addition, the singular forms, “a,” “an,” and “the” are intended to include the plural forms as well, unless context clearly indicates otherwise.

Herein, a numerical value related to a certain component is construed to include a tolerance range in interpretation of constituent components, unless clearly stated otherwise.

As used herein to represent a specific numerical range, “X to Y” means “greater than or equal to X and less than or equal to Y”.

### Composition for Preventing Hair Loss and Promoting Hair Growth

A composition for preventing hair loss and promoting hair growth according to one embodiment of the present invention includes: 100 parts by weight of an aqueous colloid solution containing non-ionic copper nanoparticles having an average particle diameter (D50) of about 2 nm to about 10 nm; about 1 part by weight to about 10 parts by weight of glycerin; and about 1 part by weight to about 10 parts by weight of a co-solvent, wherein the non-ionic copper nanoparticles are present in a concentration of about 1,500 ppm to about 2,500 ppm.

The aqueous colloid solution containing about 1,500 ppm to about 2,500 ppm of the non-ionic copper nanoparticles having an average particle diameter (D50) of about 2 nm to about 10 nm may be prepared by a method for preparing the composition for preventing hair loss and promoting hair growth described below. Advantageously, the non-ionized copper nanoparticles) (Cu⁰) having an average particle diameter (D50) of about 2 nm to about 10 nm (hereinafter referred to as “non-ionic copper nanoparticles”) can prevent hair loss while increasing the thickness of hair or promoting hair growth upon contact with the scalp and does not have side effects even after use for a long period of time.

If the average particle diameter (D50) of the copper nanoparticles exceeds the above range, it is difficult for the copper nanoparticles to be absorbed into the skin pore due to scalp sebum and the copper nanoparticles are likely to remain as waste on the scalp.

In addition, if the concentration of the non-ionic copper nanoparticles in the aqueous colloid solution is less than about 1,500 ppm, the composition exhibits insignificant effects in prevention of hair less and boosting hair growth, and if the concentration of the non-ionic copper nanoparticles exceeds about 2,500 ppm, there is a high concern of side effects on the scalp when the composition is used for a long period of time

The glycerin is a colorless, odorless, highly viscous liquid and serves to improve skin moisturizing power of the scalp by securing excellent skin moisturizing properties upon contact with the scalp.

The glycerin may be present in an amount of about 1 part by weight to about 10 parts by weight, specifically about 3 parts by weight to about 8 parts by weight, relative to 100 parts by weight of the aqueous colloid solution. Within this range, the glycerin can improve skin moisturizing power of the scalp without deterioration in effects of preventing hair loss and promoting hair growth.

The co-solvent may include at least one selected from the group of methanol, ethanol, propanol, butanol, ethyl acetate, propylene glycol, and butylene glycol. Specifically, ethanol may be used as the co-solvent in consideration of compatibility with the aqueous colloid solution and solubility of other components.

The co-solvent may be present in an amount of about 1 part by weight to about 10 parts by weight, specifically about 3 parts by weight to about 8 parts by weight, relative to 100 parts by weight of the aqueous colloid solution. Within this range, the co-solvent can maximize compatibility between the components of the composition while securing sterilization effects.

In another embodiment, the aqueous colloid solution may further include silver nanoparticles. The silver nanoparticles can interfere with metabolism of bacteria through antibacterial activity to suffocate the bacteria and perform strong sterilization activity while enhancing body's immunity.

The silver nanoparticles may have an average particle diameter (D50) of about 50 nm to about 150 nm, specifically about 70 nm to about 100 nm. Within this range, the silver nanoparticles can exhibit a sufficient antibacterial effect without deterioration in other effects.

The silver nanoparticles may be present in a concentration of about 20 ppm to about 200 ppm in the aqueous colloid solution. Within this range, the silver nanoparticles can maximize antibacterial activity without deterioration in the effects of preventing hair loss and promoting hair growth.

In the aqueous colloid solution, the copper nanoparticles and the silver nanoparticles may be present in a concentration of about 10:1 to about 50:1, specifically about 15:1 to about 40:1, specifically about 20:1 to about 38:1. Within this range, the aqueous colloid solution can secure good balance between the effects of preventing hair loss and promoting hair growth and antibacterial activity.

In another embodiment, the composition may further include a combined extract of Coicis semen and Rehmannia glutinosa. Coicis semen extract serves to prevent hair loss by promoting proliferation of dermal papilla cells while interfering with 5α-reductases, and Rehmannia glutinosa extract serves to promote hair growth.

The combined extract of Coicis semen and Rehmannia glutinosa may be present in an amount of about 0.01 parts by weight to about 3 parts by weight in the composition for preventing hair loss and promoting hair growth. Within this range, the combined extract exhibits good effects in prevention of hair loss and boosting hair growth without side effects.

The combined extract of Coicis semen and Rehmannia glutinosa may be extracted using a solvent containing about 90 wt % to about 99 wt % of ethanol and about 1 wt % to about 10 wt % of glycerin, and Coicis semen and Rehmannia glutinosa may be present in a weight ratio of about 1:2 to about 1:20. Advantageously, the solvent containing ethanol and glycerin has high extraction efficiency with respect to the combined extract of Coicis semen and Rehmannia glutinosa.

In addition to the components described above, the composition for preventing hair loss and promoting hair growth may further include typical additives, such as fats, organic solvents, solubilizers, thickening agents, gelling agents, softening agents, antioxidants, suspending agents, stabilizers, foaming agents, fragrances, surfactants, water, ionic or nonionic emulsifiers, fillers, metal ion blockers, chelating agents, preserving agents, vitamins, blockers, wetting agents, essential oils, dyes, pigments, hydrophilic or lipophilic activation agents, lipid vesicles, and external agents. Here, the additives may be present in an amount of about 0.01 parts by weight to about 3 parts by weight in the composition.

### Method for Preparing Composition for Preventing Hair Loss and Promoting Hair Growth

Another aspect of the present invention relates to a method for preparing a composition for preventing hair loss and promoting hair growth. In one embodiment, the method for preparing a composition for preventing hair loss and promoting hair growth may include: preparing an aqueous colloid solution containing non-ionic copper nanoparticles having an average particle diameter (D50) of about 2 nm to about 10 nm; and adding glycerin and a co-solvent to the prepared aqueous colloid solution, followed by uniformly mixing the glycerin and the co-solvent with the aqueous colloid solution, wherein the aqueous colloid solution is prepared by adding sodium hydroxide (NaOH) to an aqueous copper chloride (CuCl₂) solution to generate copper oxide and copper hydroxide in the aqueous solution and adding hydrazine (N₂H₄) to the aqueous solution to reduce the copper oxide and the copper hydroxide into non-ionic copper nanoparticles.

The aqueous colloid solution may be prepared by a wet type reduction process, which includes: adding sodium hydroxide (NaOH) to the aqueous copper chloride (CuCl₂) solution to generate copper oxide and copper hydroxide in the aqueous solution and adding hydrazine (N₂H₄) to the aqueous solution to reduce the copper oxide and the copper hydroxide into copper nanoparticles.

According to the present invention, copper chloride (CuCl₂) is used as a precursor of the copper nanoparticles. Unlike copper sulfate (CuSO₄), copper chloride (CuCl₂) has an anion functional group having relatively high electronegativity and thus has a different anion effect than sulfate ions in the solution, thereby further suppressing aggregation of the prepared particles with each other. Accordingly, the method according to the present invention allows preparation of finer particles while securing excellent control of surface morphology.

The step of adding sodium hydroxide (NaOH) to the aqueous copper chloride (CuCl₂) solution to generate copper oxide and copper hydroxide in the aqueous solution is a process in which sodium hydroxide (NaOH) is added to the aqueous copper chloride (CuCl₂) solution to generate copper oxide (CuO), which is an intermediate compound, and copper hydroxide (Cu(OH)₂), which is a complex. This reaction may be represented by Reaction Formula 1.

3CuCl₂+6NaOH→CuO+6Na⁺+6Cl+Cu₂O+Cu(OH)₂+2H₂O [Formula 1]

In Reaction Formula 1, sodium hydroxide (NaOH) serves to generate the copper oxide and the copper hydroxide through separation of chlorine from copper atoms of copper chloride (CuCl₂) and may be added in an amount of about 1 mole to about 6 moles per mole of copper chloride. If the content of sodium hydroxide exceeds about 6 moles, the aqueous solution turns into a strong base, which causes inefficient reduction of hydrazine added later, a large amount of unreacted products can be generated, thereby deteriorating economic feasibility, and residual ions can increase in the aqueous solution, thereby increasing impurities. If the content of sodium hydroxide (NaOH) is less than 1 mole, the form of copper oxide (CuₓO) as an intermediate compound is not completed, thereby making it difficult to achieve efficient reaction.

When sodium hydroxide (NaOH) is added to the aqueous copper chloride (CuCl₂) solution, the aqueous copper chloride solution may be adjusted to a temperature of about 25° C. to about 60° C. If the temperature of the aqueous copper chloride solution is less than about 25° C., it is difficult to form the intermediate compound, and if the temperature of the aqueous copper chloride solution exceeds about 60° C., the intermediate compound can be generated and agglomerated at an excessively rapid rate and reduction reaction is carried out at an excessively high temperature, thereby causing deterioration in thermal stability of the intermediate compound.

In the step of adding hydrazine (N₂H₄) to the aqueous solution to reduce the copper oxide and the copper hydroxide into the copper nanoparticles, the copper nanoparticles)(Cu⁰) are precipitated in a non-ionic state through reduction of the copper oxide (CuO), which is an intermediate compound, and the copper hydroxide (Cu(OH)₂), which is a complex, by adding hydrazine (N₂H₄) thereto. This reaction may be represented by Reaction Formula 2.

CuO+6Na⁺+6Cl⁻+Cu₂O+Cu(OH)₂+2N₂H₄→3Cu+6Na+6Cl⁻+4H₂O+2N_{2 [}Reaction Formula 2]

In Reaction Formula 2, hydrazine (N₂H₄) is added in an amount of about 1 mole to about 12 moles per mole of copper chloride. If the content of hydrazine is less than about 1 mole, reduction can become incomplete, and if the content of hydrazine exceeds about 12 moles, severe aggregation of the copper nanoparticles can occur due to use of an excess of hydrazine, despite rapid reduction.

When hydrazine (N₂H₄) is added to the aqueous solution, the temperature of the aqueous solution is preferably maintained at about 35° C. to about 60° C. If the temperature of the aqueous solution is less than about 35° C., complete reduction occurs at a low rate and at a low conversion rate, thereby making it difficult to achieve complete reduction. Conversely, if the temperature of the aqueous solution exceeds about 60° C., severe aggregation of the copper nanoparticles can occur due to reduction at high temperature, despite increase in reduction rate.

After preparation of the aqueous colloid solution by the method described above, glycerin and a co-solvent are added to the aqueous solution, followed by uniformly mixing the glycerin and the co-solvent with the aqueous solution.

The glycerin and the co-solvent are substantially the same as those described in the composition for preventing hair loss and promoting hair growth.

The aqueous colloid solution may contain about 1,500 ppm to about 2,500 ppm of the non-ionic copper nanoparticles. Here, sodium hydroxide (NaOH) may be added in an amount of about 1 mole to about 6 moles per mole of copper chloride (CuC12) and hydrazine (N2H4) may be added in an amount of about 1 mole to about 12 moles per mole of copper chloride (CuC12).

In order to adjust the particle size of the copper nanoparticles, the concentration of the copper chloride or the content of a reductant may be adjusted. However, increase in concentration of the copper chloride results in increase in concentration of the aqueous colloid solution and if the concentration of the copper chloride in the aqueous colloid solution exceeds about 2,500 ppm, it is difficult to control the particle size of the copper nanoparticles to about 10 nm or less and the particle size of the copper nanoparticles can be increased due to loss of stability and aggregation of the copper nanoparticles.

The aqueous colloid solution may further contain about 20 ppm to about 200 ppm of silver nanoparticles having an average particle diameter (D50) of about 50 nm to about 150 nm, specifically about 70 nm to about 100 nm. The silver nanoparticles serve to interfere with metabolism of bacteria through antibacterial activity to suffocate the bacteria and perform strong sterilization activity while boosting the immune system. Within this range, the silver nanoparticles can maximize antibacterial activity without deterioration in the effects of preventing hair loss and boosting hair growth.

In the aqueous colloid solution, the copper nanoparticles and the silver nanoparticles may be present in a concentration ratio of about 10:1 to about 50:1, specifically about 15:1 to 40:1, more specifically about 20:1 to about 38:1. Within this range, the aqueous colloid solution can secure good balance between the effects of preventing hair loss and promoting hair growth and antibacterial activity.

The method for preparing the composition for preventing hair loss and promoting hair growth may further include: uniformly mixing a combined extract of Coicis semen and Rehmannia glutinosa extracted using a solvent containing about 90 wt % to about 99 wt % of ethanol and about 1 wt % to about 10 wt % of glycerin with the aqueous colloid solution.

The combined extract of Coicis semen and Rehmannia glutinosa is substantially the same as the combined extract of the composition for preventing hair loss and promoting hair growth described above.

The composition for preventing hair loss and promoting hair growth according to the present invention may be prepared in a formulation of hair tonic, hair conditioner, hair essence, hair lotion, hair shampoo, hair rinse, hair treatment, hair nutrition cream, hair wax, hair pack, hair soap, hair mousse or hair spray. In addition, the composition may be used as an external preparation for skin in the form of a liquid, cream, paste or solid phase.

### Mode for Invention

Next, the present invention will be described in more detail with reference to some examples. It should be understood that these examples are provided for illustration only and are not to be in any way construed as limiting the invention.

Descriptions of details apparent to those skilled in the art will be omitted for clarity.

### EXAMPLE

### Example 1

100 ml of a 2M aqueous copper chloride (CuCl₂) solution was heated to 35° C. and maintained at this temperature while strongly stirring the solution. While maintaining the aqueous copper chloride solution at this temperature, 6M of sodium hydroxide (NaOH) was added all at once to the aqueous solution. After addition of the sodium hydroxide (NaOH) to the aqueous solution, 15M hydrazine (N₂H₄) was added all at once to the aqueous solution while maintaining the temperature of the aqueous solution at 45° C., thereby preparing an aqueous colloid solution through reduction of copper particles. The aqueous colloid solution contained 2,000 ppm of the copper nanoparticles. Then, silver nanoparticles having an average particle diameter (D50) of 100 nm were added to the aqueous solution until the silver nanoparticles had a concentration of 100 ppm.

Relative to 100 parts by weight of the aqueous colloid solution, 2 parts by weight of glycerin and 3 parts by weight of ethanol were added to the aqueous colloid solution and uniformly mixed therewith to prepare a composition for preventing hair loss and promoting hair growth.

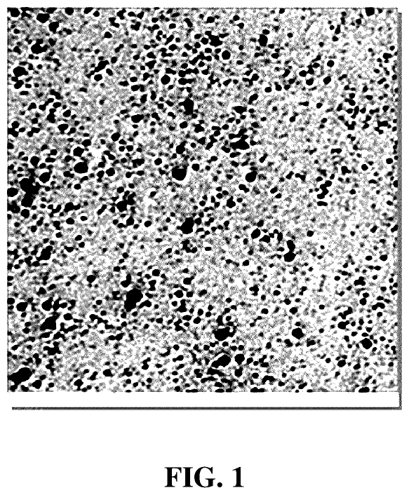

In the finally prepared composition for preventing hair loss and promoting hair growth, the copper nanoparticles had an average particle diameter (D50) of 3 nm. FIG. 1 is a scanning electron microscope (SEM) image of the non-ionic copper nanoparticles contained in the composition for preventing hair loss and promoting hair growth prepared in Example 1.

### Example 2

A composition for preventing hair loss and promoting hair growth was prepared in the same manner as in Example 1 except that 0.1 kg of Coicis semen and 0.4 kg of Rehmannia glutinosa were added to 10 L of a solvent containing 94 wt % of ethanol and 6 wt % of glycerin, stirred therewith at room temperature for 3 days, left at 2° C. for 5 day, and filtered through a filtration net, thereby providing 1 part by weight of an extract, which in turn was stirred together with 2 parts by weight of glycerin and 3 parts by weight of ethanol. Here, Coicis semen and Rehmannia glutinosa were added in a weight ratio of about 1:10.

### Comparative Example 1

A composition for preventing hair loss and promoting hair growth was prepared in the same manner as in Example 1 except that an aqueous solution containing 100 ppm of silver nanoparticles having an average particle diameter (D50) of 100 nm was used instead of the aqueous colloid solution of Example 1.

### Comparative Example 2

A composition for preventing hair loss and promoting hair growth was prepared in the same manner as in Example 2 except that an aqueous solution containing 100 ppm of silver nanoparticles having an average particle diameter (D50) of 100 nm was used instead of the aqueous colloid solution of Example 2.

### Comparative Example 3

A composition for preventing hair loss and promoting hair growth was prepared in the same manner as in Example 1 except that a 4M aqueous copper chloride (CuC12) solution was used. Here, the aqueous colloid solution contained 3,700 ppm of the copper nanoparticles.

### Comparative Example 4

A composition for preventing hair loss and promoting hair growth was prepared in the same manner as in Example 2 except that a 4M aqueous copper chloride (CuCl₂) solution was used. Here, the aqueous colloid solution contained 3,700 ppm of the copper nanoparticles.

Experimental Example—Confirmation of Hair Growth Effect and Measurement of Variation in Diameter of Hair

Six persons with male pattern alopecia among men and women aged 20 years or older were recruited and each of the compositions for preventing hair loss and promoting hair growth prepared in Examples 1 and 2 and Comparative Examples 1 to 4 was tested for 60 days by spraying the composition onto a hair loss area of each of the recruited persons three times a day. An increase rate of the number of hairs per 1 cm² (1 cm×1 cm) and an increase rate of hair thickness before and after testing were measured and results are shown in Table 1.

TABLE 1

| | | Example | | Comparative Example | | | |
|---|---|---|---|---|---|---|---|
| | | 1 | 2 | 1 | 2 | 3 | 4 |
| Number | Before test | 70 | 65 | 72 | 68 | 65 | 70 |
| of hairs | (number) | | | | | | |
| | After test | 77 | 75 | 74 | 67 | 63 | 73 |
| | (number) | | | | | | |
| | Increase | 10.0 | 15.3 | 2.78 | −1.5 | −3.0 | 4.3 |
| | rate (%) | | | | | | |
| Thickness | Before test | 0.058 | 0.055 | 0.059 | 0.050 | 0.055 | 0.062 |
| of hair | (mm) | | | | | | |
| | After test | 0.062 | 0.060 | 0.058 | 0.052 | 0.056 | 0.060 |
| | (mm) | | | | | | |
| | Increase | 6.9 | 9.1 | −1.7 | −4 | 1.8 | −3.2 |
| | rate (%) | | | | | | |

As shown in Table 1, it could be seen that the composition for preventing hair loss and promoting hair growth according to the present invention increased not only the number of hairs but also the thickness of hair, whereas the compositions prepared without containing the copper nanoparticles according to the present invention or prepared using the copper nanoparticles not in the concentration range of the present invention failed to achieve the advantageous effects of the present invention.

Although some exemplary embodiments have been described herein, it should be understood by those skilled in the art that these embodiments are given by way of illustration only, and that various modifications, variations and alterations can be made without departing from the spirit and scope of the invention. Therefore, the embodiments and the accompanying drawings should not be construed as limiting the technical spirit of the present invention, but should be construed as illustrating the technical spirit of the present invention.

1. A composition for preventing hair loss and promoting hair growth, comprising:
100 parts by weight of an aqueous colloid solution containing non-ionic copper nanoparticles having an average particle diameter (D50) of about 2 nm to about 10 nm;
about 1 part by weight to about 10 parts by weight of glycerin; and
about 1 part by weight to about 10 parts by weight of a co-solvent,
wherein the non-ionic copper nanoparticles are present in a concentration of about 1,500 ppm to about 2,500 ppm in the aqueous colloid solution.

2. The composition according to claim 1, wherein the aqueous colloid solution further contains about 20 ppm to about 200 ppm of silver nanoparticles.

3. The composition according to claim 2, wherein the copper nanoparticles and the silver nanoparticles are present in a concentration ratio of about 10:1 to about 50:1.

4. The composition according to claim 1, further comprising:
about 0.01 parts by weight to about 3 parts by weight of a combined extract of Coicis semen and Rehmannia glutinosa.

5. A method for preparing a composition for preventing hair loss and promoting hair growth, the method comprising:
preparing an aqueous colloid solution containing non-ionic copper nanoparticles having an average particle diameter (D50) of about 2 nm to about 10 nm; and
adding glycerin and a co-solvent to the prepared aqueous colloid solution, followed by uniformly mixing the glycerin and the co-solvent with the aqueous colloid solution,
wherein the aqueous colloid solution is prepared by adding sodium hydroxide (NaOH) to an aqueous copper chloride (CuCl₂) solution to generate copper oxide and copper hydroxide in the aqueous solution, and adding hydrazine (N₂H₄) to the aqueous solution to reduce the copper oxide and the copper hydroxide into non-ionic copper nanoparticles.

6. The method according to claim 5, wherein the aqueous colloid solution comprises about 1,500 ppm to about 2,500 ppm of the non-ionic copper nanoparticles, the sodium hydroxide (NaOH) is added in an amount of about 1 mole to about 6 moles per mole of copper chloride (CuCl₂), and the hydrazine (N₂H₄) is added in an amount of about 1 mole to about 12 moles per mole of copper chloride (CuCl₂).

7. The method according to claim 5, wherein the aqueous colloid solution further contains about 20 ppm to about 200 ppm of silver nanoparticles, and the copper nanoparticles and the silver nanoparticles are present in a concentration ratio of about 10:1 to about 50:1 in the aqueous colloid solution.

8. The method according to claim 5, further comprising:
uniformly mixing a combined extract of Coicis semen and Rehmannia glutinosa with the aqueous colloid solution,
the combined extract being extracted using a solvent containing about 90 wt % to about 99 wt % of ethanol and about 1 wt % to about 10 wt % of glycerin.
